(19) United States
(12) Patent Application Publication
(10) Pub. No.: US 2009/0226515 A1
(43) Pub. Date: Sep. 10, 2009

(54) STATIN COMPOSITIONS

(75) Inventors: Benoit Schmitt, Irvine, CA (US); Laurent Imler, Irvine, CA (US)

Correspondence Address: HARNESS, DICKEY & PIERCE, P.L.C., P.O. BOX 828, BLOOMFIELD HILLS, MI, 48303, US

(73) Assignee: Pharma PASS II LLC, Irvine, CA (US)

(21) Appl. No.: 12/041,976

(22) Filed: Mar. 4, 2008

(51) Int. Cl. A61K 9/20; A61K 31/40; A61P 7/00

(52) U.S. Cl. 424/465; 514/423

(57) ABSTRACT

The present invention relates to an improved statin or salt thereof composition, especially an atorvastatin composition, which exhibits enhanced dissolution properties in acidic media. It comprises a statin active ingredient and an aminoalkyl methacrylate copolymer dispersed therein, such as dimethyl aminoethyl methacrylate. The invention also relates to a process for preparing the improved composition and to methods of using it for the treatment of dyslipidemia.

### FIELD OF THE INVENTION

The invention relates to an improved statin composition, and to a process for preparing it. Especially the invention is an atorvastatin composition.

### BACKGROUND OF THE INVENTION

Statins are currently among the most therapeutically effective drugs available for reducing the level of low density lipoprotein LDL in the blood stream of a patient at risk for cardiovascular disease. Statins are also known to raise HDL cholesterol levels and decrease total triglyceride levels. Specific examples of statins include, inter alia, compactin, lovastatin, mevastatin, simvastatin, pravastatin, atorvastatin, cerivastatin, itavastatin and fluvastatin. The mechanism of action of statins has been elucidated in some detail. It is believed that statins disrupt the biosynthesis of cholesterol and other sterols in the liver by competitively inhibiting the 3-hydroxy-3-methyl-glutaryl-coenzyme A reductase enzyme (“HMG-CoA reductase”). HMG-CoA reductase catalyzes the conversion of HMG-CoA to mevalonate, which is the rate determining step in the biosynthesis of cholesterol. Consequently, its inhibition leads to a reduction in the rate of formation of cholesterol in the liver.

Pravastatin is the common name of the chemical compound [1S-[1 alpha(betaS*,deltaS*)2alpha,6alpha,8beta (R*),8aalpha]]-1,2,6,7,8,8a-hexahydro-beta,delta,6-trihydroxy-2-methyl-8-(2-methyl-1-oxobutoxy)-1-naphthaleneheptanoic acid monosodium salt, disclosed in U.S. Pat. No. 4,346,227 to Terahara et al. Pravastatin sodium is an HMG-CoA reductase inhibitor. Pravastatin is sold in the U.S. under the trademark PRAVACHOL®.

Atorvastatin calcium, another HMG-CoA reductase inhibitor, is described in U.S. Pat. No. 5,273,995 to Roth. Atorvastatin calcium is [R—(R*,R*)-2-(4-fluorophenyl)-beta,delta-dihydroxy-5-(1-methylethyl)-3-phenyl-4-[(phenyl amino)-carbonyl]-1H-pyrrole-1-heptanoic acid, hemicalcium salt. Atorvastatin calcium is sold in the U.S. under the trademark LIPITOR®.

Patent U.S. Pat. No. 7,030,151 deals with compositions which have therapeutic equivalence of atorvastatin calcium in a pharmaceutical form or comprised in a composition or a pharmaceutical formulation regardless of the form. However, this goal is achieved by atorvastatin calcium forms providing a pH equal to or greater than the pKa+1 of atorvastatin calcium.

U.S. Pat. No. 6,558,659 and companion RE39,502 to Fox discloses stabilized compositions of ring-opened 7-substituted-3,5-dihydroxyheptanoic acid or ring-opened 7-substituted-3,5-dihydroxyheptenoic acid, said composition comprising a polymer comprising an amino group or an amido group. Preferred polymers are polyinylpyrrolidone (especially cross-linked PVP), polynoxylin and cholestyramine. These polymers are said to confer stability to these statin compositions.

Eudragit® polymers are well-known in the pharmaceutical industry, and are used typically as coatings for tablets. US2005/0277691 discloses modified release statin composition comprising an outer coating applied as a membrane. Eudragit® polymers are mentioned as possible coating materials.

These statins compositions of the prior art have however the drawback of showing a poor solubility in an acidic medium such as the gastric juice. The dissolution is thus poor and the resulting bioavailability is reduced.

### SUMMARY OF THE INVENTION

The invention relates to a stating or salt thereof composition comprising at least one aminoalkyl methacrylate copolymer dispersed therein, especially wherein the vastatin or salt thereof is atorvastatin.

According to one aspect of the present invention, the composition is a vastatin composition comprising between 10 and 50 wt % of dimethyl aminoethyl methacrylate, especially wherein the vastatin or salt thereof is atorvastatin.

According to another aspect of the present invention, the statin or salt thereof composition has dissolution, for a composition containing 80 mg of equivalent statin, in a 1000 ml USP HCl buffer at pH 2.2, using the paddles method at 75 rpm, of at least 40% at 10 minutes, at least 50% at 20 minutes and at least 60% at 60 minutes. Equivalent statin refers to the weight of the drug expressed as statin.

According to one embodiment of the present invention, the statin or salt thereof composition has a dissolution, for a composition containing 80 mg of equivalent statin, in a 1000 ml USP HCl buffer at pH 2.2, using the paddles method at 75 rpm, of at least 60% at 10 minutes, at least 70% at 20 minutes and at least 80% at 60 minutes.

According to another embodiment of the invention, the atorvastatin or salt thereof composition has a dissolution, for a composition containing 80 mg of equivalent statin, in a 1000 ml USP HCl buffer at pH 2.2, using the paddles method at 75 rpm, of at least 40% at 10 minutes, at least 50% at 20 minutes and at least 60% at 60 minutes.

According to another embodiment of the invention, the atorvastatin or salt thereof composition has a dissolution, for a composition containing 80 mg of equivalent statin, in a 1000 ml USP HCl buffer at pH 2.2, using the paddles method at 75 rpm, of at least 60% at 10 minutes, at least 70% at 20 minutes and at least 80% at 60 minutes.

The invention also relates to a method of treatment of dyslipidemia, comprising the step of orally administering to a patient in need of such treatment a therapeutically effective unit dosage of a statin or salt thereof composition comprising at least one aminoalkyl metahcrylate copolymer dispersed therein.

According to another embodiment of the present invention, the method comprises the step of orally administering to a patient in need of such treatment a therapeutically effective effective unit dosage of statin or salt thereof composition having a dissolution, for a composition containing 80 mg of equivalent statin, in a 1000 ml USP HCl buffer at Ph 2.2, using the paddles method at 75 rpm, of at least 40% at 10 minutes, at least 50% at 20 minutes, and at least 60% at 60 minutes, especially wherein the statin or salt thereof is atorvastatin.

The present invention also relates to a method for the manufacture of a stating composition, comprising the steps of (i) preparing a granule comprising a statin and at least one aminoalkyl methacrylate copolymer mixed therewith, and (ii) compressing the resulting granule into a tablet.

Apart from allowing stability, the invention allows achieving high solubility in the acidic medium of the stomach, which in turn allows plasmatic concentration sufficient to achieve the therapeutical effects.

### BRIEF DESCRIPTION OF THE DRAWINGS

FIG. 1 is a representation of the solubility of compositions of the invention and a composition of the prior art in an acidic medium.

### DETAILED DESCRIPTION OF EMBODIMENTS OF THE INVENTION

The term “statin” as used in the invention designates at least one HMG-CoA reductase inhibitor. Preferably the statin is at least one ring-opened 7-substituted-3,5-dihydroxyheptanoic acid or ring-opened 7-substituted-3,5-dihydroxyheptenoic acid. The statin is preferably selected from the group consisting of lovastatin, mevastatin, simvastatin, pravastatin, atorvastatin, cerivastatin, itavastatin, fluvastatin, pitavastatin, rosuvastatin, and salts thereof.

Preferred statins are the commercial statins, such as pravastatin, atorvastatin and fluvastatin, and especially atorvastatin.

The ring-opened 7-substituted-3,5-dihydroxy-heptanoic acid or 7-substituted-3,5-dihydroxyheptenoic acid can be used in the pharmaceutical compositions of the present invention either as the free acid or as any pharmaceutically acceptable salt thereof. The free acid can be prepared, for example, by hydrolysis of the corresponding lactone form or by treatment of the salt form of the acid with cationic exchange resin and evaporating the water portion. The free acid can be used to form the pharmaceutically acceptable salt form, by conventional methods known in the art. Among preferred pharmaceutically acceptable salts are metal and amine salts. The term “pharmaceutically acceptable metal salt” thus includes, but is not limited to, sodium, potassium, lithium, calcium, magnesium, aluminum, iron, or zinc salts. Such salts may be derived from bases such as sodium hydroxide, potassium hydroxide, lithium hydroxide, calcium hydroxide, 1-deoxy-2-(methylamino)-D-glucitol, magnesium hydroxide, zinc hydroxide, aluminum hydroxide, ferrous or ferric hydroxide, ammonium hydroxide, salts formed by reaction with ammonium hydroxide or organic amine salt or for example methylglucamine, choline, arginine, 1-deoxy-2-(methylamino)-D-glucitol and the like. A preferred salt is a metal salt such as the sodium or calcium salt.

The amount of the active ingredient in the pharmaceutical compositions of the present invention will be a therapeutically effective amount. A therapeutically effective amount will generally be an amount within the range of from about 0.05 to about 70%, and preferably an amount within the range of from about 1 to about 60% by weight of the composition. It is understood that higher or lower weight percentages of the active ingredient may be present in the pharmaceutical compositions. By “therapeutically effective amount” as used herein is meant an amount of active component in the pharmaceutical compositions of the present invention which is effective to beneficially treat the patient in need thereof. An especially adapted range for the active ingredient content is 10 to 40% by weight.

The second component of the composition of the invention is the aminoalkyl methacrylate copolymer which is dispersed therein. Such polymer is a known polymer and is available as the Eudragit®E100 from Rohm & Haas. The inventor has found that surprisingly, when the polymer is used dispersed in the composition instead of used as a coating, a composition with enhanced solubility in acidic medium is obtained.

Said polymer is preferably a dimethyl aminoethyl methacrylate, and preferably is Eudragit®E1100.

The pharmaceutical compositions of the present invention will generally contain Eudragit polymer between about 10 to about 70% by weight percent; preferably between about 10 to about 50 wt %; and even more preferably between 20 and 40 wt % by weight of the pharmaceutical composition. A particularly preferred percentage of the Eudragit polymer is about 30%.

The invention thus provides a pharmaceutical composition having an enhanced solubility in acidic medium and thus a better bioavailability. Preferably, the composition exhibits a dissolution in a 1000 ml USP HCl buffer at pH 2.2, using the paddles method at 75 rpm, of at least 40% at 10 minutes, at least 50% at 20 minutes and at least 60% at 60 minutes, and more preferably of at least 60% at 10 minutes, at least 70% at 20 minutes and at least 80% at 60 minutes.

Such values could not be reached with the formulations of the prior art.

The preferred dosage forms of the pharmaceutical compositions of the present invention are solid dosage forms adapted for oral administration. Tablet dosage forms are the particularly preferred solid dosage forms of the stabilized pharmaceutical compositions of the present invention. Tablet dosage forms may contain for example, as excipients, any pharmaceutically acceptable lubricant, binder, disintegrant, diluent, carrier, preservative or combination thereof. Solid dosage forms that are not formulated as tablets typically do not need a lubricant component since this is typically added to facilitate manufacture of tablet dosage forms. For the purpose of oral preparations of the present invention, pharmaceutically acceptable inert carriers or diluent or filler can be either solid or not. Among other preferred dosage forms useful for formulating the stabilized pharmaceutical compositions of the present invention include powders, dispersible granules, capsules and cachets.

The pharmaceutical compositions of the present invention may also contain any pharmaceutically acceptable excipient or combination thereof. Conventional pharmaceutical excipients include those which function in a dosage form, for example, as a lubricant, glidant, diluent, binder, disintegrant, carrier, colorant, preservative or coating material. Examples of pharmaceutically acceptable excipients include, but are not limited to, lactose, sugar, saccharose, corn starch, hydrolyzed starch (malto-dextrine), modified corn starch, mannitol, sorbitol, silicon dioxide, microcrystalline cellulose, croscarmellose sodium, polyvinylpyrrolidone, polyvinylalcohol, hydroxypropylcellulose, hydroxymethylcellulose, hydroxypropylmethylcellulose, methylcellulose, ethylcellulose, gelatin, cross-linked PVP, sodium carboxymethyl starch, magnesium stearate, sodium stearyl fumarate, polyethylene glycol, stearic acid, hydrogenated vegetable oil, glyceyl behenate and talc, and mixtures thereof.

Further excipients are disclosed in “Handbook of Pharmaceutical excipients”, 2^{nd} (Ed., 1994, American Pharmaceutical Association, Washington, ISBN 0 91730 66 8, by Wade A., Weller P J.).

The pharmaceutical compositions may be manufactured by various techniques available to the skilled man.

One preferred process comprises the steps of preparing granules of the active together with the methacrylic polymer, and then compressing said granules with extra-granular excipients, into a tablet. Granules manufacture can take place according to various techniques, such fluidized bed technology, tank mixing, or crushing compacts previously manufactured. Granules are optionally, but not necessarily, compressed into tablets. One embodiment includes the steps of dry mixing the components, compressing into a compressed form and crushing said compressed form into granules.

When the granule obtained (whether subsequently coated or not) is compressed to form tablets, this step can be implemented using any conventional technique which is suitable, for example using alternating or rotating compressing machines.

Direct compression is also available for the manufacture of the instant pharmaceutical compositions.

The compositions of the invention may further contain a stabilizing amount of a basifying agent such as disclosed in U.S. Pat. No. 5,180,589, U.S. Pat. No. 5,686,104 or a buffering agent such as disclosed in WO-A-00/35425. Examples of such stabilizers are inorganic metal oxides and hydroxides, especially magnesium oxide, aluminum oxide, alkali metal hydroxides such as sodium hydroxide, potassium hydroxide or lithium hydroxide and alkaline earth metal hydroxides such as calcium hydroxide or magnesium hydroxide, and any basic inorganic pharmaceutically acceptable calcium, magnesium, aluminum or lithium salt, or mixtures thereof. Examples of these salts are calcium carbonate, calcium hydroxide, magnesium carbonate, magnesium hydroxide, magnesium silicate, magnesium aluminate, aluminum magnesium hydroxide, lithium hydroxide or calcium hydroxide, or mixtures thereof; and buffers such as sodium or potassium citrate, sodium phosphate, dibasic sodium phosphate, calcium carbonate, hydrogen phosphate, phosphate, sulphate, sodium or magnesium carbonate, sodium ascorbinate, benzoate, sodium or potassium hydrogen carbonate, lauryl sulphate, or mixtures thereof.

The pharmaceutical compositions of the present invention may also contain any pharmaceutically acceptable organic acids such as malic acid or tartaric acid. Amounts by weight of release enhancer can be selected in the range of 0.05 to 30%, preferably, 10 to 20% and more preferably between 10 and 15%.

The invention also provides methods for the treatment of dyslipidemia, comprising the step of orally administering to a patient in need of such treatment a therapeutically effective unit dosage form of the invention.

### EXAMPLES

The following examples illustrate the invention without limiting it.

### Example 1

The following formulations are prepared.

| | | Amount (mg) | | | | | | | |
|---|---|---|---|---|---|---|---|---|---|
| Excipients | Function | I | | II | | III | | IV | |
| Atorvastatin | API | 82.9 | | 82.9 | | 82.9 | | 82.9 | |
| Calcium | | | | | | | | | |
| Eudragit ® E100 | Solubility | 120.0 | | 80.0 | | 160.0 | | — | |
| | enhancer | | | | | | | | |
| Croscarmellose | Disintegrant | 30.0 | | 40.0 | | 100.00 | | 60.0 | |
| Sodium | | | | | | | | | |
| Lactose | Filler | 150.0 | | 120.0 | | 160.0 | | 280.0 | |
| monohydrate | | | | | | | | | |
| Glyceryl Behenate | Lubricant | 8.0 | | 8.1 | | 10.0 | | 10.0 | |
| Colloidal Silicon | Glidant | 2.0 | | 2.0 | | 2.0 | | 2.0 | |
| Dioxide | | | | | | | | | |
| | Total (mg) | 392.9 | mg | 333.0 | mg | 514.9 | mg | 434.9 | mg |

Atorvastatin Calcium, Eudragit® E100, Lactose and Croscarmellose are dry blended, then compacted together. The compact mass is then crushed in granules. These granules are blended with Glyceryl behenate and colloidal silicon dioxide, and compressed into tablets.

Dissolution tests are carried out, in an apparatus according to the US Pharmacopeia, XXIV, type II paddle, at 75 rpm, in a dissolution medium comprised of 1000 ml of USP HCl buffer pH 2.2.

The results (expressed in dissolved fraction in %) are the following.

% dissolved

| Time (min) | I | II | III | IV |
|---|---|---|---|---|
| 0 | 0 | 0 | 0 | 0 |
| 10 | 81 | 59 | 81 | 17 |
| 20 | 88 | 68 | 86 | 26 |
| 30 | 90 | 71 | 88 | 32 |
| 40 | 91 | 73 | 88 | 36 |
| 50 | 91 | 75 | 89 | 39 |
| 60 | 91 | 76 | 89 | 41 |

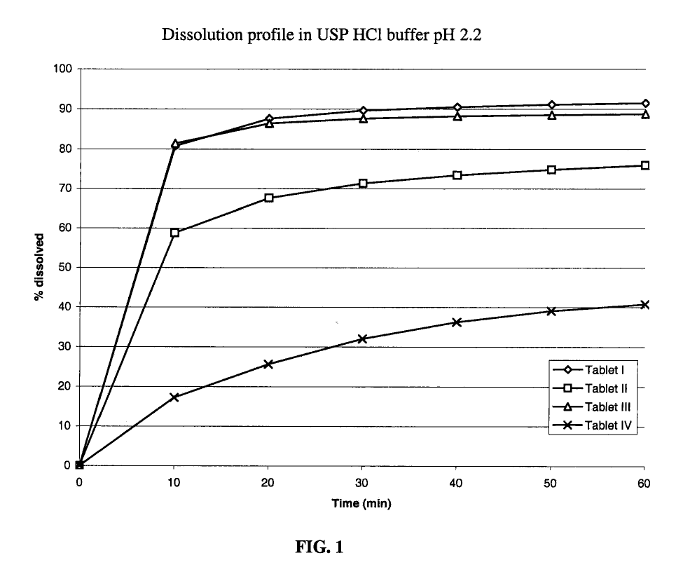

Results clearly show that the dissolution in the case of the invention is far different from the prior art. FIG. 1 is a representation of these results.

### Example 2

Stability tests were conducted on the compositions according to the prior art (Lipitor®) and according to the invention with the formulation of example 1. Two series of tests were performed at storage conditions of 40° C. and 75% of relative humidity, and assays after 1, 2 and 3 months of the impurities and of the effective amount left in the composition.

| | | Initial | 1 months | 2 months | 3 months |
|---|---|---|---|---|---|
| | | % wt of impurities | | | |
| 40° C. 75% | Composition of example | 2.05% | 2.27% | 2.77% | 2.86% |
| RH | I, without Eudragit ®. | | | | |
| 40° C. 75% | Composition of example | 1.85% | 2.01% | 2.33% | 2.53% |
| RH | I, with Eudragit ® core. | | | | |
| 40° C. 75% | Lipitor ® | 0.40% | 0.40% | 0.50% | 0.49% |
| RH | | | | | |
| | | % wt of effective amounts remaining | | | |
| 40° C. 75% | Composition of example | 82.6 | 80.00 | 80.60 | 80.90 |
| RH | I, without Eudragit. | | | | |
| 40° C. 75% | Composition of example | 84.1 | 83.40 | 83.20 | 80.10 |
| RH | I, with Eudragit core. | | | | |
| 40° C. 75% | Lipitor ® | 89.12 | 89.60 | 87.82 | 89.80 |
| RH | | | | | |

Eventhough the tests of the composition according to the invention show a slightly lower stability than the Lipitor® composition, the example with a Eudragit polymer within the core show acceptable stability.

| | | #5973 | #5974 | #5975 | #5976 | #5979 | #5980 | #5983 | #5984 |
|---|---|---|---|---|---|---|---|---|---|
| Tablet composition | Atorvastatin Ca | 82.9 | 82.9 | 82.9 | 82.9 | 82.9 | 82.9 | 82.9 | 82.9 |
| | Eudragit E | — | 120.0 | — | 120.0 | — | 120.0 | — | 120.0 |
| | Croscarmellose | 30.0 | 30.0 | 30.0 | 30.0 | 30.0 | 30.0 | 30.0 | 30.0 |
| | Na | | | | | | | | |
| | Lactose | 150.0 | 150.0 | 150.0 | 150.0 | 150.0 | 150.0 | 150.0 | 150.0 |
| | Sodium Stearyl | 6.0 | 6.0 | 6.0 | 6.0 | 6.0 | 6.0 | 6.0 | 6.0 |
| | Fumarate | | | | | | | | |
| | Colloidal | 2.0 | 2.0 | 2.0 | 2.0 | 2.0 | 2.0 | 2.0 | 2.0 |
| | Silicon Dioxide | | | | | | | | |
| | Magnesium | — | — | 15.0 | 15.0 | — | — | — | — |
| | Oxide | | | | | | | | |
| | Magnesium | — | — | — | — | — | — | — | — |
| | Carbonate | | | | | | | | |
| | Magnesium | — | — | — | — | 25.0 | 25.0 | — | — |
| | Hydroxide | | | | | | | | |
| | Sodium | — | — | — | — | — | — | — | — |
| | Bicarbonate | | | | | | | | |
| | Calcium | — | — | — | — | — | — | 50.0 | 50.0 |
| | Phosphate | | | | | | | | |
| | Dibasic | | | | | | | | |
| pH in 900 ml | After 15′ | | 3.25 | | 3.63 | | 3.37 | | 3.75 |
| HCl 0.001N | After 30′ | | 3.26 | | 3.85 | | 3.53 | | 3.90 |
| | Impurities | 2.23 | 1.78 | 1.80 | 1.73 | 1.73 | 1.49 | 1.91 | 1.72 |
| | increase after 1 | | | | | | | | |
| | month at 40° c. | | | | | | | | |
| | 75% RH in % | | | | | | | | |

Further comparative tests were conducted on different compositions according to the invention (comprising the Eudragit component) and comprising further stabilizers. Results showed acceptable improved stability for the compositions according to the invention.

1. A statin or salt thereof composition having a dissolution, for a composition containing 80 mg of equivalent statin, in a 1000 ml USP HCl buffer at pH 2.2, using the paddles method at 75 rpm, of at least 40% at 10 minutes, at least 50% at 20 minutes and at least 60% at 60 minutes.

2. The composition of claim 1 having a dissolution in a 1000 ml USP HCl buffer at pH 2.2, using the paddles method at 75 rpm, of at least 60% at 10 minutes, at least 70% at 20 minutes and at least 80% at 60 minutes.

3. The composition of claim 1, comprising between 10 and 40% of statin.

4. The composition of claim 1, wherein the statin is at least one ring-opened 7-substituted-3,5-dihydroxyheptanoic acid or ring-opened 7-substituted-3,5-dihydroxyheptenoic acid.

5. The composition of claim 1, wherein the statin is selected from the group consisting of lovastatin, mevastatin, simvastatin, pravastatin, atorvastatin, cerivastatin, itavastatin, fluvastatin, pitavastatin, rosuvastatin, and salts thereof.

6. The composition of claim 1, wherein the statin is under a salt form.

7. The composition of claim 1, under a tablet form.

8. The composition of claim 1, wherein the statin or salt thereof is atorvastatin.

9. The composition of claim 2, wherein the statin or salt thereof is atorvastatin.

10. The composition of claim 8, under a tablet form.

11. A statin or salt thereof composition comprising at least one aminoalkyl methacrylate copolymer dispersed therein.

12. The composition of claim 11, wherein the aminoalkyl methacrylate copolymer is dimethyl aminoethyl methacrylate.

13. The composition of claim 11, comprising between 10 and 50 wt % of aminoalkyl methacrylate copolymer.

14. The composition of claim 11, comprising between 20 and 40 wt % of aminoalkyl methacrylate copolymer.

15. The composition of claim 11, comprising between 10 and 40% of statin.

16. The composition of claim 11, wherein the statin is at least one ring-opened 7-substituted-3,5-dihydroxyheptanoic acid or ring-opened 7-substituted-3,5-dihydroxyheptenoic acid.

17. The composition of claim 11, wherein the statin is selected from the group consisting of lovastatin, mevastatin, simvastatin, pravastatin, atorvastatin, cerivastatin, itavastatin, fluvastatin, pitavastatin, rosuvastatin, and salts thereof.

18. The composition of claim 11, wherein the statin is under a salt form.

19. The composition of claim 11, under a tablet form.

20. The composition of claim 11, wherein the statin or salt thereof is atorvastatin.

21. The composition of claim 20, comprising between 10 and 40% of atorvastatin.

22. The composition of claim 20, under a tablet form.

23. A method for the treatment of dyslipidemia, comprising the step of orally administering to a patient in need of such treatment a therapeutically effective unit dosage of a statin or salt thereof composition comprising at least one aminoalkyl methacrylate copolymer dispersed therein.

24. A method for the treatment of dyslipidemia, comprising the step of orally administering to a patient in need of such treatment a therapeutically effective unit dosage of statin or salt thereof composition having a dissolution, for a composition containing 80 mg of equivalent statin, in a 1000 ml USP HCl buffer at pH 2.2, using the paddles method at 75 rpm, of at least 40% at 10 minutes, at least 50% at 20 minutes and at least 60% at 60 minutes.

25. A method for the manufacture of a statin composition, comprising the steps of (i) preparing a granule comprising a statin and at least one aminoalkyl methacrylate copolymer mixed therewith, and (ii) compressing the resulting granule into a tablet.
